(19) United States
(12) Patent Application Publication
(10) Pub. No.: US 2009/0192471 A1
(43) Pub. Date: Jul. 30, 2009

(54) DISPOSABLE INFUSION DEVICE WITH LOCKED CANNULA

(75) Inventors: Brett J. Carter, Monroe, WA (US); Clifton A. Alferness, Port Orchard, WA (US); John M. Adams, Snohomish, WA (US); Daniel Hawkins, Newcastle, WA (US)

Correspondence Address: GRAYBEAL JACKSON LLP, 155 - 108TH AVENUE NE, SUITE 350, BELLEVUE, WA, 98004-5973, US

(73) Assignee: Calibra Medical, Inc., Redwood City (CA)

(21) Appl. No.: 12/418,929

(22) Filed: Apr. 6, 2009

Related U.S. Application Data
(63) Continuation of application No. 11/641,596, filed on Dec. 18, 2006.

(51) Int. Cl. A61M 5/14

(52) U.S. Cl. 604/180

(57) ABSTRACT

An infusion assembly comprises a disposable wearable infusion device having a body arranged to be adhered to a patient's skin and a cannula arranged to be received by the device body to deploy the cannula for delivering a medicament to beneath the patient's skin. A lock locks the cannula within the device body when the cannula is deployed in the device.

### CLAIM OF PRIORITY

This present application is a continuation of co-pending U.S. patent application Ser. No. 11/641,596, filed on Dec. 18, 2006 which is incorporated by reference in its entirety.

### BACKGROUND OF THE INVENTION

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses (boluses) for meals and snacks. These infusion systems have shown to improve control of blood glucose levels. However, they suffer the drawbacks of size, cost, and complexity. For example, these pumps are electronically controlled and must be programmed to supply the desired amounts of basal and bolus insulin. This prevents many patients from accepting this technology over the standard subcutaneous injections.

Hence, there is a need in the art for a convenient form of insulin treatment which does not require significant basal and bolus needs. Preferably, such a treatment would be carried out by an infusion device that is simple to use and mechanically driven negating the need for batteries and the like. It would also be preferable if the infusion device could be directly attached to the body and not require any electronics to program the delivery rates. The insulin is preferably delivered through a small, thin-walled tubing (cannula) through the skin into the subcutaneous tissue similar to technologies in the prior art.

While the idea of such a simple insulin delivery device is compelling, many obstacles must be overcome before such a device may become a practical realty. One problem resides in insulin supply. Patients vary greatly on the amount of insulin such a device must carry to provide treatment over a fixed time period of, for example, three days. This is one environment where one size does not fit all. Another problem is with cannula deployment to support insulin delivery. Cannula deployment to support delivery of the insulin beneath the patient's skin must be made easy and convenient. This is not as easy as it seems because cannula deployment, as generally and currently performed in the art, requires insertion of a cannula carrying needle into the patient and then retraction of only the needle to leave the cannula in place beneath the patient's skin. As will be seen subsequently, the present invention addresses these and other issues toward providing a simple, practical, and reliable insulin delivery device.

### SUMMARY OF THE INVENTION

The invention provides an infusion assembly comprising a disposable wearable infusion device having a body arranged to be adhered to a patient's skin, a cannula arranged to be received by the device body to deploy the cannula for delivering a medicament to beneath the patient's skin, and a lock that locks the cannula within the device body when the cannula is deployed in the device.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a schematic representation of a reservoir within a disposable wearable diffusion device being filled with a liquid medicant according to an embodiment of the present invention;

FIG. 2 is perspective view of an infusion device embodying the present invention;

FIG. 3 is a perspective view of the device of FIG. 2 with the device rotated 180 degrees;

FIG. 4 is a side view, with portions cut away, of a service device embodying the present invention with an infusion device detachably received therein and about to receive a vial of liquid medicant;

FIG. 5 is side view, with portions cut away, of the service device of FIG. 4 after having partially received the vial of liquid medicant for venting the interior of the vial to atmospheric pressure in accordance with an embodiment of the present invention;

FIG. 6 is a side view, with portions cut away, of the service device of FIG. 4 after having fully received the vial of liquid medicant to establish fluid communication between the vial and the infusion device in accordance with an embodiment of the present invention;

FIG. 7 is a side view, with portions cut away, of the service device of FIG. 4 after having provided the infusion device with a desired quantity of the liquid medicant from the vial;

FIG. 8 is a side view, with portions cut away, of the service device of FIG. 4 after having been placed in a cannula drive configuration to an enable a cannula driver in accordance with an embodiment of the present invention;

FIG. 9 is a side view, with portions cut away, of the service device of FIG. 4 after the cannula driver has driven a cannula carried on an inner needle to a cannula deployment position;

FIG. 10 is a side view, with portions cut away, of the service device of FIG. 4 after the cannula driver has withdrawn the needle upon which the cannula was driven from the cannula leaving the cannula behind in a deployed position;

FIG. 11 is a side view, with portions cut away, of the service device of FIG. 4 after the infusion device and the service device are separated leaving the infusion device deployed on the skin of the patient with the cannula extending beneath the patient's skin in a deployed position to deliver the liquid medicant to the patient;

FIG. 12 is a perspective view of the device of FIGS. 2 and 3 illustrating the relation of the cannula to the device before cannula deployment;

FIG. 13 is a partial sectional side view of a cannula assembly and device according to an embodiment of the invention;

FIG. 14 is a partial sectional view of the cannula assembly and device of FIG. 13 showing the cannula during deployment;

FIG. 15 is a partial sectional view of the cannula assembly and device of FIG. 13 showing the cannula in a deployed position;

FIG. 16 is a partial sectional view of the cannula assembly and device of FIG. 13 showing the cannula in a deployed position with the needle withdrawn;

FIG. 17 is a partial sectional side view of another cannula assembly and device according to an embodiment of the invention;

FIG. 18 is a partial sectional view of the cannula assembly and device of FIG. 17 showing the cannula during deployment;

FIG. 19 is a partial sectional view of the cannula assembly and device of FIG. 17 showing the cannula in a deployed position;

FIG. 20 is a partial sectional view of the cannula assembly and device of FIG. 17 showing the cannula in a deployed position with the needle withdrawn;

FIG. 21 is a perspective view of an infusion device and cannula driver according to an embodiment of the invention;

FIG. 22 is a cross-sectional view of the infusion device and cannula driver of FIG. 21 with the cannula driver detachably received on the infusion device and ready to deploy a cannula;

FIG. 23 is a sectional view similar to FIG. 22 but illustrating the cannula being deployed; and

FIG. 24 is a sectional view similar to FIG. 22 illustrating the cannula driver and infusion device after the cannula has been deployed.

### DETAILED DESCRIPTION OF THE INVENTION

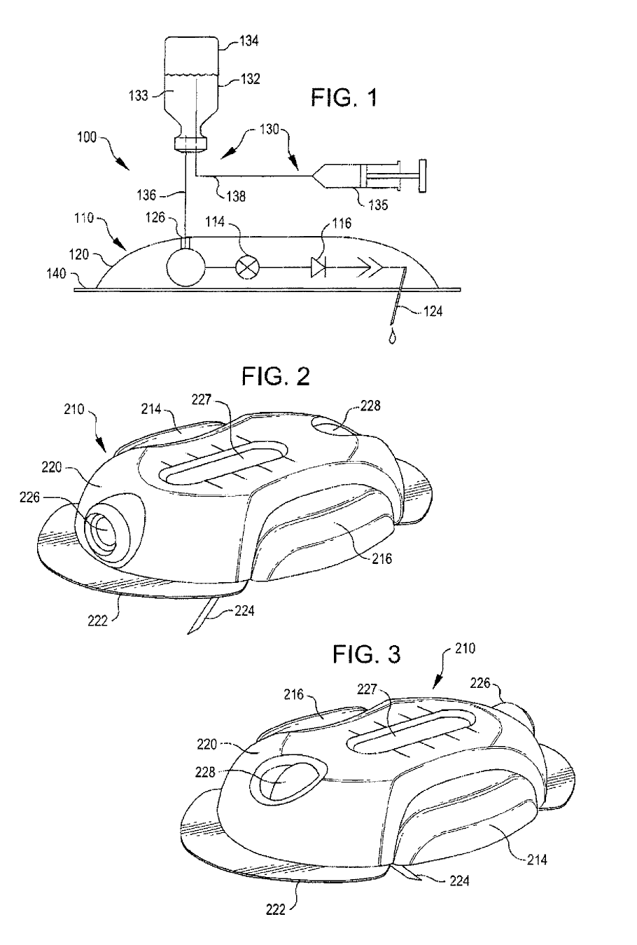

Referring now to FIG. 1, it is a schematic representation of an infusion system embodying the present invention. The system 100 generally includes a disposable wearable infusion device 110 and a filler 130. The filler 130 is seen in FIG. 1 filling a reservoir 112 within the diffusion device 110 with a measured quantity of a liquid medicant, such insulin, for example, according to an embodiment of the present invention.

The device 110 includes a body or enclosure 120 that is adhered to the skin 140 of a patient and that encloses the reservoir 112. The device 130 further includes a cannula 124 deployed from the device 110 to beneath the skin 140 of a patient to deliver the liquid medicant. The reservoir 112 is coupled to the cannula 124 by a pump 114 and a one-way check valve 116. Actuation of the pump provides a fixed quantity of the medicant to the cannula.

The filler 130 is adapted to receive a vial 132 of the liquid medicant 133. A first conduit 136 provides fluid communication from the vial 132, through a filling port septum 126, and into the reservoir 112. A second conduit 138 provides fluid communication from a pump 135 to the vial. The pump 135 is employed to pump air into the vial 132 to displace a known quantity of the liquid medicant 133 from the vial 132. The medicant is then delivered to the reservoir through the first conduit 136. As will be seen subsequently, and in accordance with one aspect of the present invention, as the vial 132 is received by the filler 130, the interior space 134 within the vial 132 is vented to atmospheric pressure and then sealed. This venting of the vial 132 assures that a known volume of air being pumped into the vial will displace a like volume of medicant for filling the reservoir.

Once the reservoir 112 is filled with a desired quantity of liquid medicant, the infusion device may be adhered to the patient's skin 140. Preferably thereafter, the cannula 124 is deployed.

Referring now to FIG. 2, it is perspective view of an infusion device 210 embodying the present invention. FIG. 3 is a perspective view of the device 210 after being rotated 180 degrees. The device 210 includes an enclosure 220 and a base 222. The device 210 further includes a pair of actuator buttons 214 and 216 which, when concurrently depressed, cause a fixed quantity of liquid medicant to be dispensed from a cannula 224. The device 210 further includes a filler port septum 226 through which the reservoir (not shown) is filled and a viewing window 227 through which the liquid medicant may be viewed during the filling process. More particularly, the viewing window provides a means by which air bubbles within the reservoir may be seen to facilitate removal thereof. Lastly, as may be seen in FIGS. 2 and 3, the device 210 includes an auxiliary port septum 228. The port septum 228 is provided to enable deployment of the cannula 224 in a manner as described subsequently and to receive boluses of liquid medicant, such as basal or long acting insulin, to be dispensed through the cannula 224.

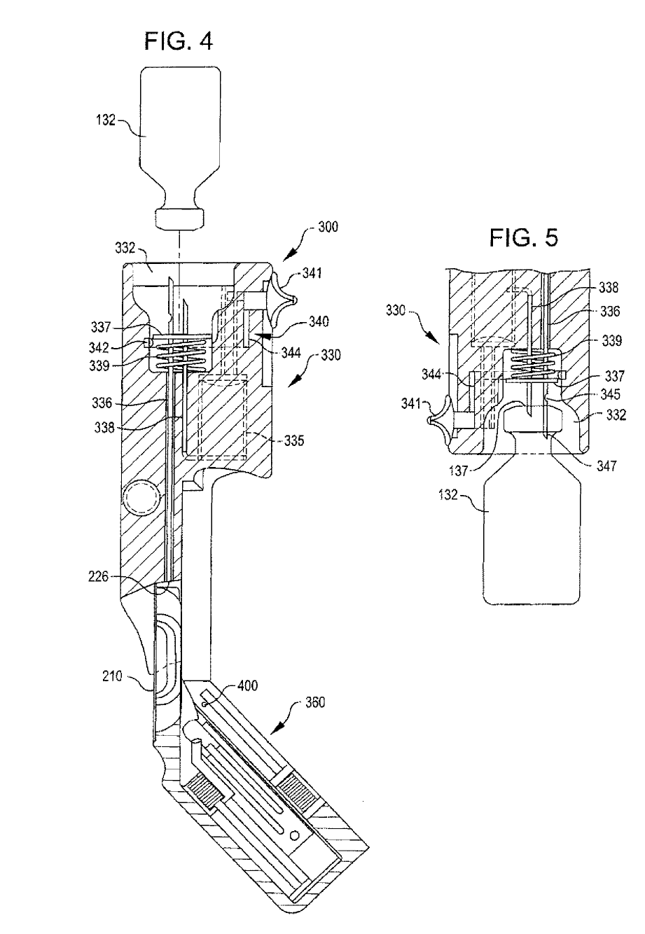

FIG. 4 is a side view, with portions cut away, of a service device 300 embodying the present invention with the infusion device 210 detachably received therein. The service device includes a filler device 330 and a cannula driver 360. The filler device 330 and cannula driver 360, for reasons which will become apparent herein after, are pivotally connected at a pivot point 400.

As seen in FIG. 4, the filler device 330 is about to receive a vial 132 of liquid medicant. The filler device 330 includes a cavity 332 for receiving the vial 132. The filler device 330 further includes a first conduit and a second conduit 338. As described in connection with FIG. 1, the first conduit serves to provide fluid communication between the vial 132 and the reservoir (not shown) of the infusion device 210 to be filled. The second conduit provides fluid communication between the pump 335 within the filler device 330 and the vial 132. The first conduit 336 is carried on a stop 337 which is spring loaded by a spring 339. As will be seen subsequently, when the vial 132 engages stop 339, further movement of the vial into the cavity 332 will cause the first conduit 336 to travel with the stop 339 and vial 132 forcing the end of the first conduit 336 into the filling port septum 226.

The filler device 330 also includes an interlock 340 that prevents the pump arm from being displaced and thus premature actuation thereof before the vial 132 is fully received within the cavity. Hence, the filler device is enabled to transfer a volume of the liquid medicant to the infusion device reservoir only upon the cavity 332 fully receiving the vial 132. To that in end, the interlock 340 includes a follower 342 and an inter connected latch 344. As shown in FIG. 4, the latch 344 prevents the arm 341 from being displaced. However, when the vial is received into the cavity 332, the follower engages the vial 132 and is displaced causing the latch to be pulled free of the arm 341. This operation will be seen more clearly herein after.

FIG. 5 shows how the vial 132 may be vented as it is received into the cavity of the filler device 330. In FIG. 5 it may be seen that the vial 132 has a sealing membrane 137. Also, it may be seen that the first conduit 226 includes an opening 345 displaced from the end 347 of the first conduit 336. To vent the vial 132, the vial 132 and the filler device 330 are inverted as illustrated. When the vial 132 is advanced to cause the membrane to be pierced by the end 347 of the first conduit 336, the vial 132 is vented to atmospheric pressure as long as the membrane 137 is between the end 347 and the opening 345 of the first conduit 336.

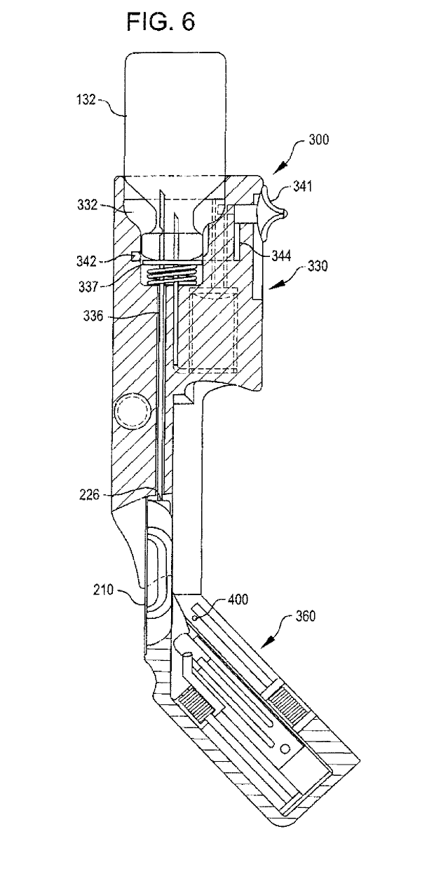

FIG. 6 shows the vial 132 fully received within the cavity 332 of the filler device 330. The follower 342 has been displaced by its engagement with the vial 132 to cause the latch 344 to be moved clear of the arm 341. Also, the first conduit 336 has entered the filling port septum 226 of the infusion device 210 after having traveled with the stop 337 and the vial 132. The infusion device 210 is now ready to be filled with a quantity of liquid medicant from the vial 132.

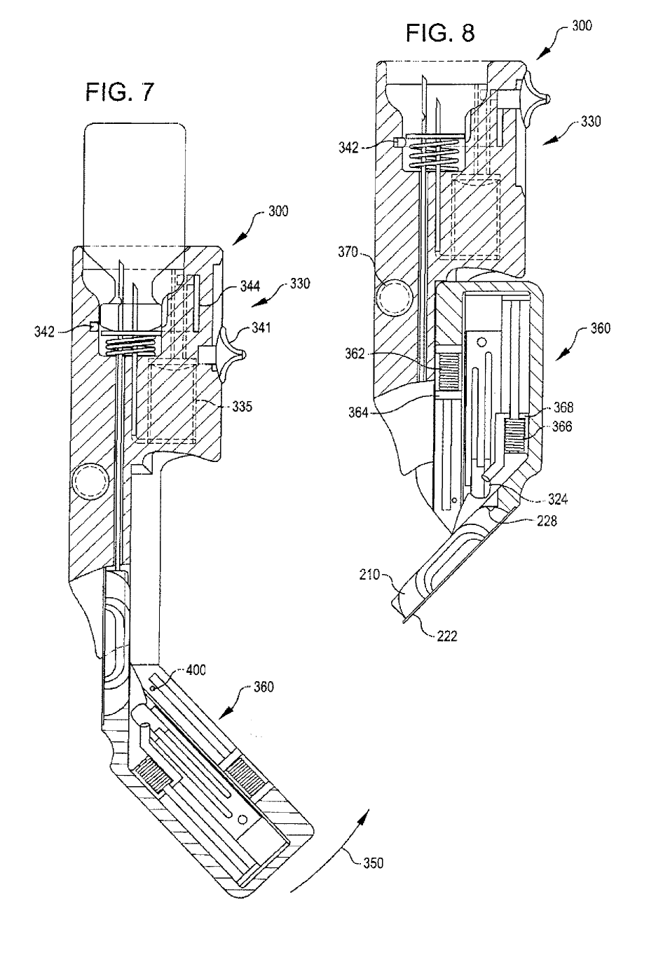

FIG. 7 shows the filler device 330 after having filled the infusion device 210 with a quantity of liquid medicant. The quantity of liquid medicant filled depends upon the length of travel of the arm 341 and hence the amount of air pumped into the vial 132 by the pump 335. In this manner, the quantity of liquid medicant filled may be accurately metered. Further, after the liquid medicant has been transferred to the infusion device 210, the medicant may be viewed through the window 227 (FIGS. 2 and 3) for air bubbles. Any observed air bubbles may be eliminated by dithering the arm 341 back and forth

After the filling process is completed, the vial 132 may be removed from the service device 300. Then, the cannula driver 360 is pivoted about pivot point 400 in the direction of arrow 350 (FIG. 7) and locked in the position shown in FIG. 8. With the cannula driver 360 locked in the position shown in FIG. 8, it is now in a cannula drive configuration. This results in a cannula/needle assembly 324 to be aligned with the auxiliary port septum 228 of the infusion device 210. The base 222 of the infusion device 210 may now be adhered to the patient's skin.

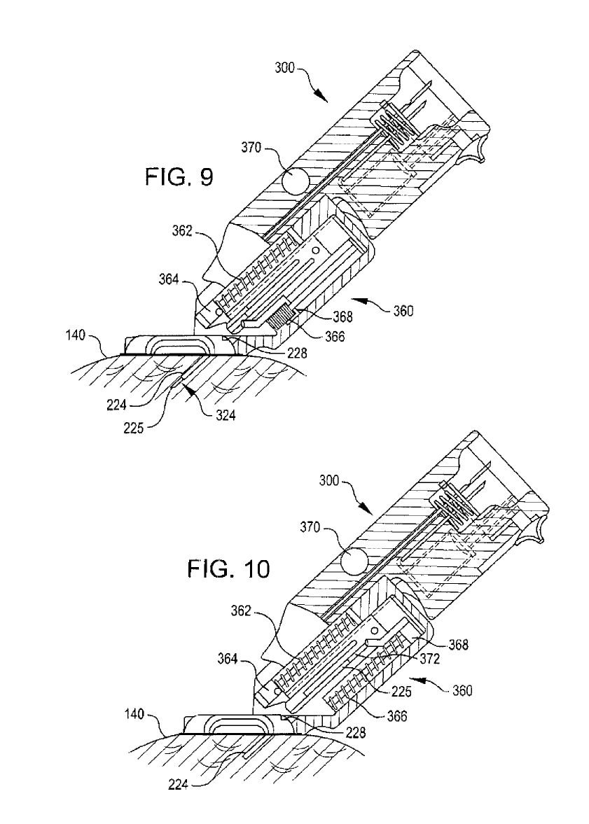

As will be seen subsequently, a first drive element 362 may now be released to drive the cannula/needle assembly 324 through the port septum 228 rendering the cannula 224 in a deployed position (FIG. 9). A second drive element 366 may thereafter be released to withdraw only the needle 225 back into the driver 360 leaving the cannula 224 in its deployed position and the needle safely tucked away for sharps disposal.

With further reference to FIG. 8, the first drive element comprises a spring. The spring 362 is coupled to a follower 364. The spring 362 may be released by depressing a pair of aligned actuator buttons on opposite sides of the driver 360. One such actuator button 370 is shown in the FIG. 8. Preferably the actuator buttons are only coupled to release the springs 362 and 366 when the driver 360 is locked in the cannula drive configuration. Hence, in this manner, the driver is enabled only when the driver is properly positioned and locked for the purpose of deploying the cannula 224.

FIG. 9 shows the driver 360 after the cannula/needle assemble 324 have been driven through the port septum 228 and the infusion device 210 to beneath the patient's skin 140. It will be noted that the first spring 362 has been released and that the follower 364 is at the end of its travel. The second spring 366 may now be released to withdraw the needle 225 from the cannula 224.

FIG. 10 shows the driver 360 after the needle has been withdrawn from the cannula 224. The needle 225 has been withdrawn back out of the infusion device 210, through the port septum 228, and into a cavity 372 within the driver 360. Hence, it will be noted that the second spring 366 has been released and that the follower 368 is at the end of its travel. With the needle 225 withdrawn from the cannula 224, the cannula 224 is left in its deployed position beneath the skin 140 of the patient while the needle 225 is safely stored for sharps disposal.

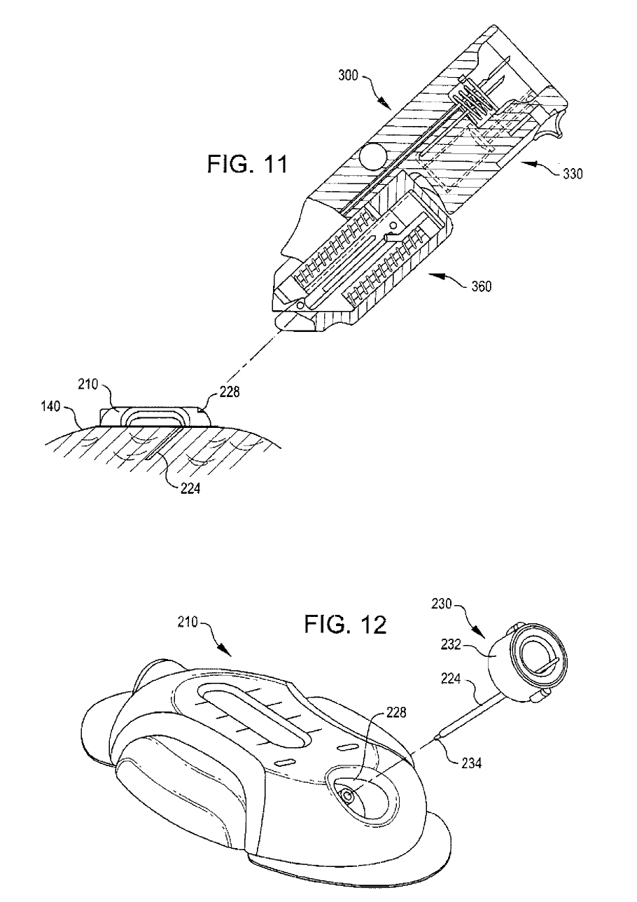

Now that the cannula 224 is deployed, the service device 300 may be removed from the infusion device 210. This is illustrated in FIG. 11. The service device 210 has been removed and separated from the infusion device 210. The infusion device 210 remains adhered to the patient's skin 140 for delivering the liquid medicant, such as insulin, to the patient through the cannula 224. The service device may be thrown away.

Referring now to FIG. 12, it is a perspective view of the device 210 of FIGS. 2 and 3 illustrating the relation of the cannula to the device before cannula deployment. As may be noted in the figure, the cannula 224 is a part of a cannula assembly 230 that further includes a cannula holder or carrier 232. The assembly 230 is carried on a cannula needle 234 which, as previously described, is withdrawn from the cannula after the cannula is deployed. The carrier 232 has a dimension to permit it to be received by the septum 228.

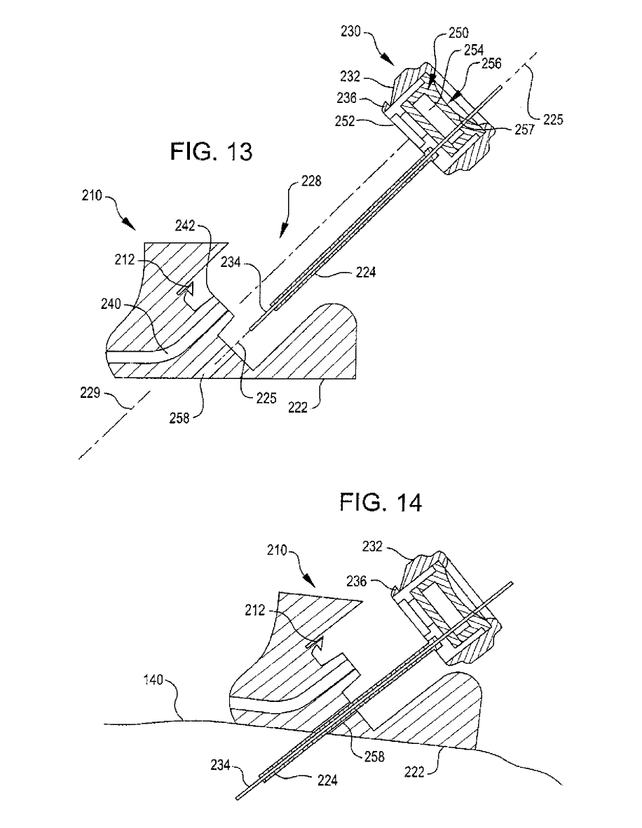

FIG. 13 is a sectional view of the cannula assembly 230 and the portion of the device 210 including the septum 228. The device 210 includes a reservoir conduit 240 that terminates at a fitting 242. The cannula carrier 232 includes a fluid coupler 250 providing fluid communication between its input 252 and the cannula 224. In this embodiment the input 252 is a port that receives the fitting 242 when the carrier is received within the septum 228 and the cannula is in a deployed position.

The fluid coupler more particularly takes the form of a chamber 254. The chamber 254 has a second input 256 that may take the form of a penetrable membrane 257. The second input 256 may be employed to receive the needle of a syringe providing boluses of insulin. The needle 234 is also withdrawn through the penetrable membrane 257 after the cannula 224 is deployed. Since the chamber 254 is in fluid communication with the cannula 224, the boluses may be delivered from the input 256 to the cannula through the chamber 254. As may be further noted in FIG. 13, the septum has a center axis 229 and the cannula 224 has a center axis 225. The center axis 225 of the cannula 224 is offset from the center axis 229 of the septum 228. This allows a much larger area of the membrane 257 to be available for receiving the needle of a syringe than would otherwise be the case if the two axes 225 and 229 were aligned.

As may be still further noted in FIG. 13, the device 210 includes a latch 212 that extends into the septum 228. The latch 212 is positioned to lockingly engage a flange 236 of the cattier 232 when the carrier 232 reaches its final position within the infusion device 210 resulting in the deployment of the cannula 224. In this manner, the carrier 232 is locked to the infusion device 210 upon the deployment of the cannula 224.

Referring now to FIG. 14, here it may be seen that during the deployment of the cannula 224, the needle 234 and cannula 224 are translated with the carrier 232 into the septum 228. The cannula 224 is further received by a channel 258 extending through the base 222 of the infusion device 210.

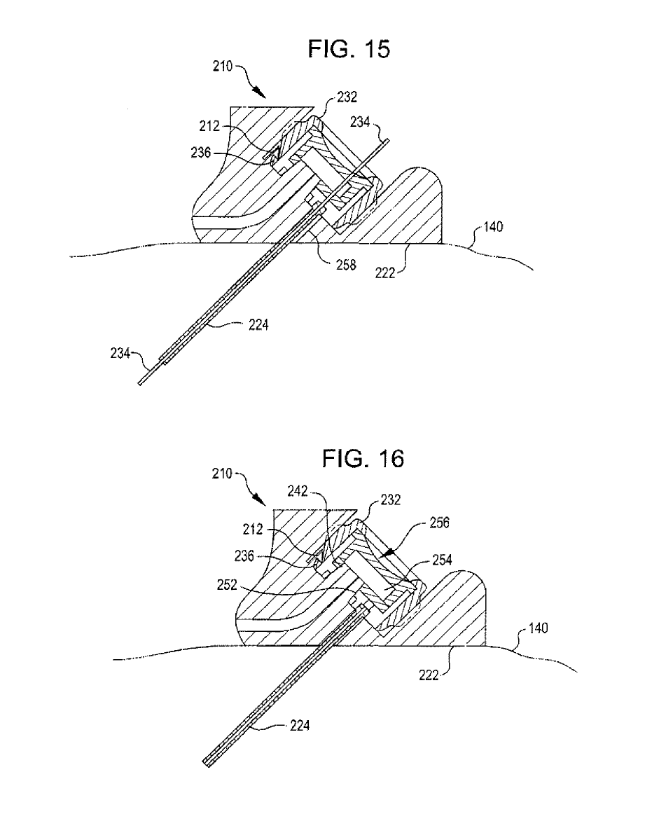

FIG. 15 shows the carrier 232 in its final position within the infusion device 210. The cannula 224 has reached its deployed position and the latch 212 has engaged the flange 236 to lock the carrier 232 within the infusion device 210. The fitting 242 has also been received by the port 252. As a result, the reservoir conduit 240 and the chamber 254 provide fluid communication between the reservoir (not shown) of the device 210 with the cannula 224. The cannula 224 now extends from the base 222 of the device 210 to beneath the skin 140 of the patient.

FIG. 16 shows the carrier 232 in its final position locked within the infusion device 210 and the cannula 224 in its deployed position after the needle 234 (FIGS. 13-15) has been withdrawn from the cannula 224. With the chamber 254 providing fluid communication between the reservoir (not shown) of the device 210 and the cannula 224, the infusion device 210 is now ready to provide measured doses of insulin to beneath the skin of the patient. Also, the second input 256 is also available for the delivery of boluses of insulin from a syringe, for example. The cannula 224 now extends from the base 222 of the device 210 to beneath the skin 140 of the patient.

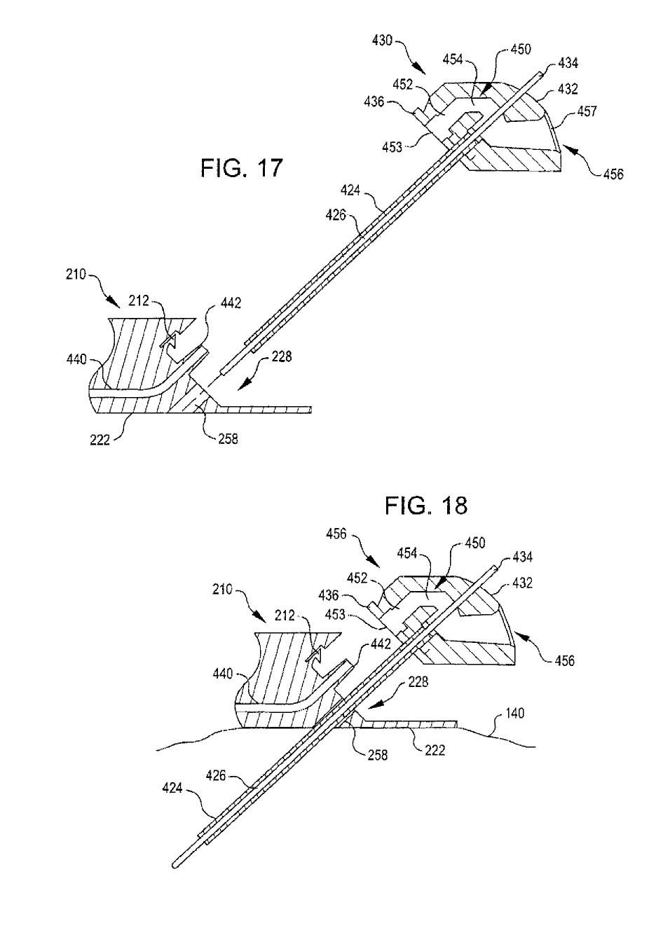

FIG. 17 is a sectional view of another cannula assembly 430 embodying the invention along that portion of the device 210 including the septum 228 which has been modified to cooperate with the cannula assembly 430. The device 210 now includes a reservoir conduit 440 that terminates at a pointed fitting 442. The cannula carrier 432 includes a fluid coupler 450 providing fluid communication between its input 452 and the cannula 424. In this embodiment the input 452 is a penetrable membrane 453 through which the fitting 442 eventually projects when the carrier 430 is received at its final position within the septum 228.

As in the previous embodiment, when the carrier 432 reaches its final position within the septum 228, the cannula 424 is in its deployed position. The carrier 432 will also be locked to the infusion device 210 by the latch 212 of the device 210 engaging the flange 436 of the carrier 432.

The fluid coupler 450 also again particularly takes the form of a chamber 454. In addition to being in fluid communication with the input 452, the chamber 454 is also in fluid communication with a second input 456 arranged to receive boluses of insulin as in the previous embodiment. Here again, the second input 456 includes a penetrable membrane 457.

As may be seen in FIG. 18, during the deployment of the cannula 424, the needle 434 and cannula 424 are translated with the carrier 432 into the septum 228. The cannula 424 is further received by a channel 258 extending through the base 222 of the infusion device 210.

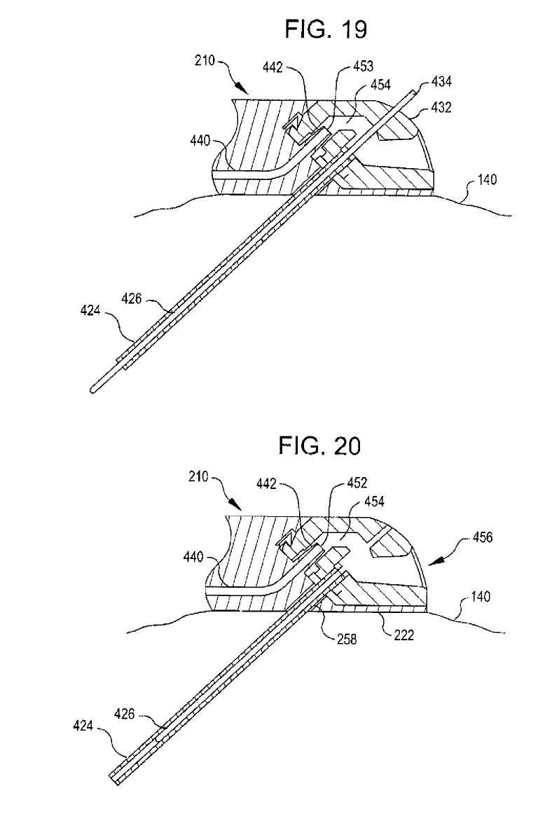

FIG. 19 shows the carrier 432 in its final position within the infusion device 210. The cannula 424 has reached its deployed position and the latch 212 has engaged the flange 436 to lock the carrier 432 within the infusion device 210. The pointed fitting 442 has also penetrated the membrane 453 (FIGS. 16 and 17) and has been received by the input 452. As a result, the reservoir conduit 440 and the chamber 454 provide fluid communication between the reservoir (not shown) of the device 210 with the cannula 424. The cannula 424 now extends from the base 222 of the device 210 to beneath the skin 140 of the patient.

FIG. 20 shows the carrier 432 in its final position locked within the infusion device 210 and the cannula 424 in its deployed position after the needle 434 (FIGS. 17-19) has been withdrawn from the cannula 424. With the chamber 454 providing fluid communication between the reservoir (not shown) of the device 210 and the cannula 424, the infusion device 210 is now ready to provide measured doses of insulin to beneath the skin of the patient. Also, the second input 456 is also available for the delivery of boluses of insulin from a syringe, for example. The cannula 424 now extends from the base 222 of the device 210 to beneath the skin 140 of the patient.

As may also be noted in FIGS. 17-20, the cannula carrier 432 further includes a cannula reinforcement structure 426 that provides the cannula 424 with an increased resistance to bending during cannula deployment. The cannula carrier 432 is thus arranged to translate the needle 434, the cannula reinforcement structure 426 and the cannula 424 to the deployed position beneath the patient's skin. The needle 434 is still arranged to be withdrawn from the cannula 424 and to be returned to the cannula driver (not shown) leaving the cannula 424 and the cannula reinforcement 426 structure in the deployed position beneath the patient's skin. The cannula reinforcement structure preferably comprises a tubular member coaxially disposed between the needle 434 and the cannula 424 and formed of metal such as stainless steel. As seen in FIG. 20, the reinforcement structure 426 is coextensive with only a portion of the cannula.

In addition to the foregoing, it may be appreciated that in each of the embodiments of FIGS. 13-20, the fitting 442 and input 452 may be reversed. In other words, the fitting 442 may be part of the carrier 432, 232, and the input 452 may be within the infusion device 210 without departing from the present invention.

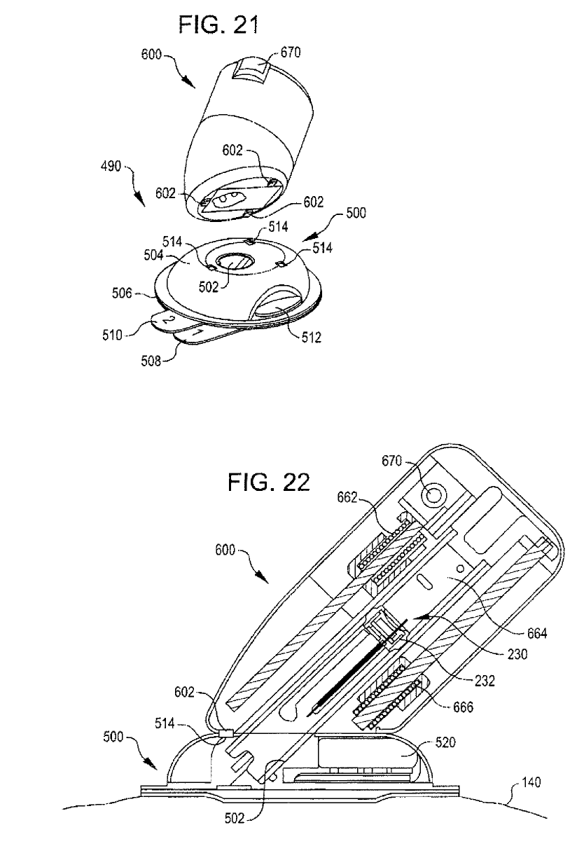

Referring now to FIG. 21, it is a perspective view of an infusion system 490 according to another embodiment of the invention. The infusion system 490 includes an infusion device 500 and a cannula driver 600. Here the device includes a single port for both receiving a cannula to be deployed and receiving boluses of insulin with a needle syringe, for example. The device includes a body 504 and a base 506. The base includes two protective strips 508 and 510. The first strip 508, when removed, uncovers an antiseptic to be applied to the skin area chosen to receive the device 500. The second strip 510, when removed, exposes a layer of adhesive for use in adhering the device 500 to the patient's skin.

As in prior embodiments, the device 500 further includes a pair of actuator buttons of which one such button 512 may be seen in FIG. 21. As before, the device 500 is preferably arranged so that only concurrent depression of the actuator buttons results in insulin being dispensed to the patient

The cannula driver 600 is arranged to detachably receive the infusion device 500 to facilitate deployment of a cannula from the device 500. To that end, the driver 600 includes a plurality of projections 602 that are arranged to align with and be frictionally received by a like plurality of recesses 514 within the body 504 of the infusion device 500. The projections 602 and the recesses 514 are correspondingly arranged to serve the further function of aligning the cannula driver 600 with the infusion device 500 for cannula deployment.

FIG. 22 is a cross-sectional view of the infusion device 500 and cannula driver 600 of FIG. 21 with the cannula driver detachably received on the infusion device and ready to deploy a cannula. Preferably, the cannula driver includes one of the cannula assemblies described herein. The cannula driver 600 of FIGS. 22-24 utilizes, for example, the cannula assembly 230 of FIGS. 13-16. Presumably, the reservoir 520 of the infusion device 500 has been already filled with a liquid medicant, such as insulin, and the device 500 has been adhered to the patient's skin 140.

As may be noted in FIG. 22, the process of detachably joining the cannula driver 600 to the infusion device 500 has aligned the port with the cannula carrier 232. As in the previous embodiment, a first drive element 662 may be released to drive the cannula/needle assembly 230 through the port septum 502 rendering the cannula 224 in a deployed position (FIG. 23). A second drive element 666 may thereafter be released to withdraw only the needle 234 back into the driver 600 leaving the cannula 224 in its deployed position and the needle 234 safely tucked away for sharps disposal.

With further reference to FIG. 22, the first drive element comprises a spring. The spring 662 is coupled to a follower 664. The spring 662 may be released by depressing a pair of aligned actuator buttons on opposite sides of the driver 600. One such actuator button 670 is shown in FIG. 22. Preferably the actuator buttons are only coupled to release the springs 662 and 666 when the driver 600 is detachably received on the infusion device 500. Hence, in this manner, the driver is enabled only when the driver is properly positioned and locked for the purpose of deploying the cannula 224.

FIG. 23 shows the driver 600 and the infusion device 500 after the cannula/needle assemble 230 has been driven through the port septum 502 and the infusion device 500 to beneath the patient's skin 140. It will be noted that the first spring 662 has been released and that the follower 664 is at the end of its travel. The second spring 666 may now be released to withdraw the needle 234 from the cannula 224.

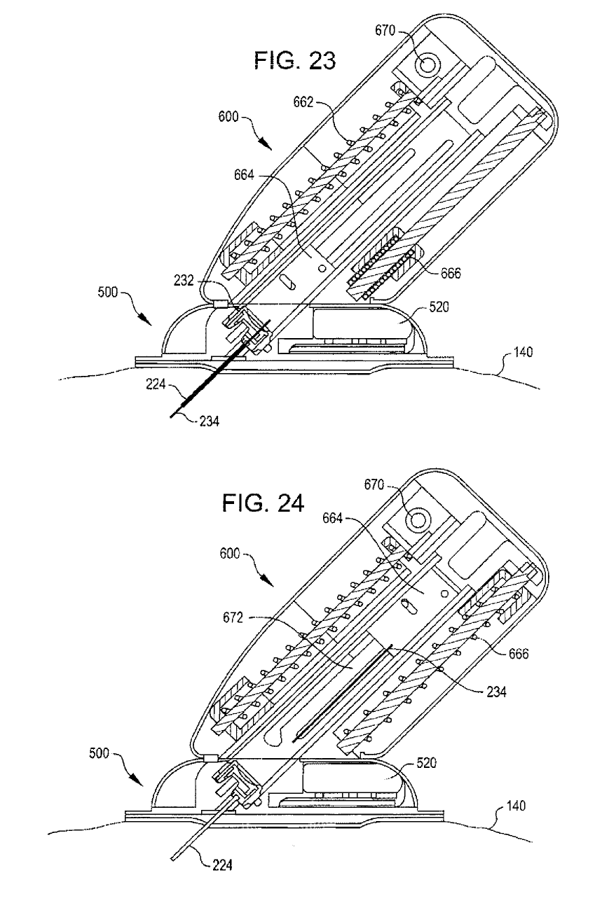

FIG. 24 shows the driver 600 and the infusion device 500 after the needle 234 has been withdrawn from the cannula 224. The needle 234 has been withdrawn back out of the infusion device 500, through the port septum 502, and into a cavity 672 within the driver 600. Hence, it will be noted that the second spring 666 has been released and that the follower 664 is at the end of its travel. With the needle 234 withdrawn from the cannula 224, the cannula 224 is left in its deployed position beneath the skin 140 of the patient while the needle 234 is safely stored for sharps disposal.

Now that the cannula 224 is deployed, the cannula driver 600 may be removed from the infusion device 500. The infusion device 500 will remain adhered to the patient's skin for delivering the liquid medicant, such as insulin, to the patient through the cannula 224.

While particular embodiments of the present invention have been shown and described, modifications may be made, and it is therefore intended in the appended claims to cover all such changes and modifications which fall within the true spirit and scope of the invention as defined by those claims.

1-43. (canceled)

44. An infusion assembly comprising:
a disposable wearable infusion device having a body arranged to be adhered to a patient's skin;
a cannula arranged to be received by the device body to deploy the cannula for delivering a medicament to beneath the patient's skin; and
a lock that locks the cannula within the device body when the cannula is deployed in the device.

45. The assembly of claim 1, wherein the lock prevents removal of the cannula from the device after having once been deployed.

46. The assembly of claim 1, wherein the lock comprises a latch.

47. The assembly of claim 3, wherein one of the cannula and the device body comprises the latch.

48. The assembly of claim 4, wherein the other one of the device body and the cannula includes a flange arranged to be engaged by the latch when the cannula is received by the device body.

49. The assembly of claim 5, wherein the device body includes a septum that receives the cannula.

50. The assembly of claim 6, further comprising a fluid coupler that couples the cannula to receive the liquid medicament when the cannula is received by the septum.

51. An infusion assembly comprising:
a disposable wearable infusion device having a body arranged to be adhered to a patient's skin and a reservoir for holding a liquid medicament to be infused into the patient;
a cannula arranged to be received by the device body to deploy the cannula for delivering the medicament within the reservoir to the patient; and
a lock that locks the cannula within the device body when the cannula is deployed in the device.

52. The assembly of claim 8, wherein the lock comprises a latch.

53. The assembly of claim 9, wherein one of the cannula and the device body comprises the latch.

54. The assembly of claim 10, wherein the other one of the device body and the cannula includes a flange arranged to be engaged by the latch when the cannula is received by the device body.

55. The assembly of claim 11, wherein the device body includes a septum that receives the cannula.

56. The assembly of claim 12, further comprising a fluid coupler that couples the cannula to receive the liquid medicament when the cannula is received by the septum.

57. The assembly of claim 8, wherein the lock prevents removal of the cannula from the device after having once been deployed.

58. An infusion assembly comprising:
a disposable wearable infusion device having a body arranged to be adhered to a patient's skin, a reservoir for holding a liquid medicament to be infused into the patient, and a septum;
a cannula subassembly including a cannula and a carrier that carries the cannula, the carrier being arranged to be received by the septum of the device to deploy the cannula, the carrier including a fluid coupler that couples the cannula to receive the liquid medicament when the cannula carrier is received by the septum; and
a lock that locks the cannula carrier within the septum when the cannula carrier is received by the septum, the lock preventing removal of the cannula after being once deployed in the septum.

59. The assembly of claim 15, wherein the lock comprises a latch.

60. The assembly of claim 16, wherein the latch is within the septum of the infusion device.

61. The assembly of claim 17, wherein the carrier has a flange arranged to be engaged by the latch when the cannula carrier is received by the septum
